(19) United States
(12) Patent Application Publication
(10) Pub. No.: US 2007/0243216 A1
(43) Pub. Date: Oct. 18, 2007

(54) Stable solutions of prostaglandin and uses of same

(76) Inventors: Stanley Kepka, (US); Wieslaw Bochenek, Newtown, PA (US)

Correspondence Address: Albert T. Keyack;Klehr, Harrison, Harvey, Branzburg & Ellers LLP, 260 South Broad Street, Philadelphia, PA, 19102, US

(21) Appl. No.: 11/404,657

(22) Filed: Apr. 14, 2006

(51) Int. Cl. A61K 31/557; A61K 9/00

(52) U.S. Cl. 424/400; 514/573; 424/402

(57) ABSTRACT

Dosage forms of a pharmaceutical composition comprising a sealed actives compartment containing a stable prostaglandin in a pharmaceutical composition for topical application to a patient are disclosed. Preferably, the prostaglandin compound is substantially uniformly dispersed in a delivery vehicle, and in certain embodiments the prostaglandin compound contains stabilizing agent comprising a moisture scavenger, an antioxidant and a chelating agent for metal ions. In certain embodiments the delivery vehicle is preferably substantially anhydrous, free of metal ions and oxygen and water. The improved compositions of the present invention are useful for treating medical conditions by providing a packed mono or multi-component dosage form that comprises a sealed section containing an active pharmaceutical ingredient and a delivery vehicle pharmaceutically compatible for topical delivery. The medical condition can be, for example male or female sexual dysfunction broadly and male erectile dysfunction specifically. Alternately, the medical condition can be a wound or other conditions such as hemorrhoids. In preferred embodiments of the medical treatment methods the active pharmaceutical ingredient is prostaglandin, and most preferably, the prostaglandin is treated with a stabilizing agent comprising a moisture scavenger, an antioxidant and a chelating agent for metal ions. In a specific application of the foregoing, a sock saturated with a gel forming material acting as lubricant is also disclosed which preferably has at least one wound healing composition to treat a foot skin condition, preferably using a wound healing composition such as prostaglandin.

The present invention relates generally to prostaglandin compositions. In particular, the present invention relates to storage stable, pharmaceutical compositions containing prostaglandins and surfactants, stabilizers and other therapeutically active additives.

### BACKGROUND OF THE INVENTION

Prostaglandins are related to prostanoic acid, which has a following structure:

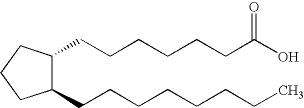

As is known in the art, prostaglandins, including those of the E type (“PGE”) and their esters, are extremely potent in causing various biological responses. For that reason, these compounds are useful for pharmacological and pharmaceutical purposes. See for example, Bergstrom et al., Pharmacol. Rev. 20, 1 (1968), and references cited therein.

Because of these biological responses, these known prostaglandins and their esters are useful to study, prevent, control, or alleviate a wide variety of diseases and undesirable physiological conditions in birds and mammals including humans, useful domestic animals, pets and zoological specimens, and laboratory animals for example mice, rats, rabbits and monkeys. For example, E-type prostaglandins are used in concentration ranges of about 10 μg/ml to about 10 mg/ml of pharmacologically suitable liquid vehicle for either topical application or as an aerosol spray.

E-type prostaglandins are also useful in the treatment of asthma. For example, these compounds are useful as bronchodilators or as inhibitors of mediators such as SRS-A and histamine, which are released from cells activated by an antigen-antibody complex. Thus, these compounds control spasms and facilitate breathing in conditions such as bronchial asthma, bronchitis, bronchiectasis, pneumonia and emphysema. For these purposes, these compounds are administrated in a variety of dosage forms, e.g., orally in the form of tablets, capsules, or liquids; rectally in the form of suppositories; parenterally, subcutaneously, or intramuscularly, with intravenous administration being preferred in emergency situations. These compounds can also be administered via inhalation in the form of aerosols or solutions for nebulizers or by insufflation in the form of powder. Doses in the range of about 0.01 mg to 5 mg per kg of body weight are used, with a frequency of 1 to 4 times per day, the exact dose depending on the age, weight, and condition of the patient and other factors. For the above uses these prostaglandins can be combined advantageously with other antiasthmatic agents, such as symphatomimetics (isoproterenol, phenylephrine, ephedrine, etc.) xanthine derivatives (theophylline and aminophylline); and corticostereoids such as prednisone. The uses of these compounds are described for example in South African Pat. No. 681,055.

E-type prostaglandins and their analogues are similarly pharmacologically useful, to reduce and control excessive gastric secretion, thereby reducing or avoiding gastrointestinal ulcer formation, and accelerating the healing of such ulcer already present in the gastrointestinal tract. For this purpose, the compound is injected intravenously, subcutaneously or intramuscularly in an infusion dose range about 0.1 μg to about 500 μg per kg of body weight per minute, or in a total daily dose by injection or infusion in the range about 0.1 mg to about 20 mg per kg of body weight per day, the exact dose depending on the weight, and condition of the patient or animal, and on the frequency and route of administration.

It is also known that E-type prostaglandins are useful whenever it is desired to inhibit platelet aggregation, to reduce the adhesive character of platelets, and to remove or prevent the formation of thrombi in mammals, including humans, rabbits, and rats. For example, these compounds are useful in the treatment and prevention of myocardial ischemia and prevention myocardial infarcts, to treat and prevent post-operative thrombosis, to promote patentcy of vascular grafts following surgery, and to treat conditions such as arteriosclerosis, blood clotting defects due to lipemia, and other clinical conditions in which the underlying etiology is associated with lipid imbalance or hyperlipidemia. For these purposes these compounds are administered systemically, e.g., intravenously, subcutaneously, intramuscularly, and in the form of sterile implants for prolonged dosages. For rapid response, especially in emergency situations, the intravenous route of administration is preferred. Dose in the range about 0.005 to about 20 mg per kg body weight per day are used, the exact dose depending on the age, weight and condition of the patient or animal, and the frequency and route of administration.

E-type prostaglandins are especially useful as additives to blood, blood products, blood substitute and other fluids which are useful in artificial extracorporeal circulation and perfusion of isolated body portions, e.g., limbs and organs, whether attached to the original body, detached and being preserved or prepared for transplant, or attached to a new body. During such circulation and perfusion aggregated platelets tends to block both blood vessels and portions of the circulation apparatus. This blockage is avoided by the presence of these compounds. For this purpose, the prostaglandin E compound is added gradually or in single or multiple portions of the circulating blood, to the blood of the donor animal, to the perfused body portion, attached or detached, to the recipient, or to two or all of those at a total steady state dose of about 0.001 10 mg per liter of circulating fluid. It is especially useful to use these compounds in laboratory animals, e.g., cats, dogs, rabbits, monkeys, and rats for these purpose in order to develop new methods and techniques for organ and limb transplants.

E-type of prostaglandin are also extremely effective for stimulation of smooth muscle cells, and are also highly active in potentiating other known smooth muscle stimulators, for example, oxytocic agents, e.g., oxytocin, and the various ergot alkaloids including derivatives and analogs thereof. Therefore, PGE₂ for example, is useful in place or in combination with less than usual amounts of these known smooth muscle stimulators, for example, to relive the symptoms of paralytic ileus, or to control or prevent atonic uterine, bleeding after abortion or delivery, to aid in expulsion of the placenta, and during the puerperium. For the latter purpose, the E-type prostaglandin is administrated by intravenous infusion immediately after abortion or delivery at a dose in the range about 0.01 to about 50 μg per kg of body weight per minute until the desired effect is obtained. Suggested doses are given by intravenous, subcutaneous, or intramuscular injection or intrafusion during puerperium in the range 0.01 to 3 mg per kg of body weight per day, the exact dose depending on the age, weight, and condition of the patient or animal. Similarly, E-type of prostaglandins are useful in place of oxytocin to induce labor in pregnant female animals including man, cows, sheep and pigs at or near term, or in pregnant animals with intrauterine death of the fetus from about 20 weeks to term. For this purpose, the compound is infused intravenously at a dose of 0.01 to 50 μg per kg body weight per minute until or near the termination of the second stage of labor, i.e., expulsion of the fetus. These compounds are especially useful when the female is one or more weeks post-mature and natural labor has not started, or 12 to 60 hours after the membrane has ruptured and natural labor ha not yet started. The alternative route of administration is oral.

One particular type of PGE, Prostaglandin E-1, is a relaxant (vasodilator) also known as Alprostadil that is used to produce erections and was originally marketed as Caverject®.

E-type prostaglandins are known to be useful as hypotensive agents to reduce blood pressure in mammals, including man. For this purpose the compounds are administrated by intravenous infusion at the rate about 0.01 to 50 g per kg of body weight per minute in single or multiple doses of about 25-500 μg per kg of body weight total per day.

E-type prostaglandins are useful for controlling the reproductive cycle in ovulating female mammals including humans and animals such as monkeys, rats, rabbits, dogs, cattle, and like. For this indication, PGE₂ for example, is administrated systemically at dose level in the range 0.001 mg to about 2 mg per kg body weight of the female mammals, advantageously during a spun of time starting approximately at the time of ovulation and ending approximately at the time of menses or just prior to menses. Intravaginal and intrauterine are alternative routs of administration. Additionally, expulsion of an embryo or a fetus is accomplished by similar administration of the compound during the first third of the normal mammalian gestation period. Another somewhat related use for E-type prostaglandins is causing cervical dilation in pregnant and non-pregnant female mammals for purpose of gynecology and obstetrics. In labor induction and clinical abortion by these compounds, cervical dilation is also observed. In cases of infertility, cervical dilation produced by PGE and PGF compounds is useful in assisting sperm movement to the uterus. Cervical dilation by prostaglandins is also useful in operative gynecology such as D and C (Cervical Dilation and Uterine Curettage) where mechanical dilation may cause perforation of the uterus, cervical tears, or infections. It is also useful in diagnostic procedures where dilation is necessary for tissue examination. For these purpose the PGE-type compound are administrated locally or systematically. PGE₂., for example, is administrated orally or vaginally at doses of about 5-50 mg per treatment of an adult female human, with from one to five treatments per 24-hour period. PGE₂ is also administrated intramuscularly or subcutaneously at doses of about one to 25 mg per treatment. The exact dosage for these purposes depends on the age, weight and condition of the patient or animal.

Finally, as mentioned before, E-type prostaglandins are potent antagonists of epinephrine-induced mobilization of free fatty acid. For this reason, these compounds are useful in experimental medicine for both in vitro and in vivo studies in mammals, including man, rabbits, and rats, intended to lead to the understanding, prevention, symptom alleviation, and cure of diseases involving abnormal lipid mobilization and high free fatty acid levels, e.g., diabetes mellitus, vascular diseases and hyperthyroidism.

Surfactants and/or solubilizers have been used with other type of drug having low water solubility. However, the addition of surfactants and /or solubilizers may enhance or adversely affect the chemical stability of drug compounds.

Prostaglandins are difficult to formulate in storage-stable solutions, as they tend to be hydrolytically unstable. In some instance, the parent adds the pharmaceutical composition of current invention, however, are storage stable. These compositions contain prostaglandin and stability-enhancing amount of polyethoxylated castor oil. U.S. Pat. No. 5,849,792. Thus, although prostaglandin compounds have wide and varied medical uses, they are invariably not suitable for storage due to their unstable nature and therefore impractical for widespread use, one factor being low solubility. Attempts to stabilize prostaglandin-E in aqueous systems by the use of cyclodextrin complexes have been reported. See, Wiese et al., J. Pharm. Science 80:153-66(1991). However, aqueous prostaglandin preparations reported as “stabilized”nonetheless have a relatively short shelf life that again limits their practical utilization. EP 330511 A2 (Ueno at al.) and EP 435682 A2 (Wheeler). These attempts have met with varying success. Surfactants and/or solubilizers have been used with other type of drug having low water solubility. However, the addition of surfactants and/or solubilizers may enhance or adversely affect the chemical stability of drug compounds.

Various other attempts have been made to improve the stability of prostaglandins using cyclodextrins, for example, formation of inclusion compound of PGs or alkyl ester thereof with cyclodextrin was disclosed in Japanese Patent Publication No. 3362/1975. An injectable preparation obtained by lyophilizing prostaglandins and cyclodextrin is disclosed in Japanese Patent Publication No.43569/1979. Injectable preparations obtained by lyophilizing prostaglandins, cyclodextrin and ascorbic acid or citric acid are disclosed in Japanese Patent Publication No. 43570/1979. Formation of inclusion compounds of PGF2 analogs with cyclodextrin was disclosed in Japanese Patent Publication No.24369/1986.

Another difficulty encountered in making prostaglandin therapeutically effective has been poor bioavilability due to their instability and anti-secretory activity in typical dose forms, as described in U.S. Pat. No. 4,310,543—Hugo-Roche

Other attempts to stabilized dosage forms of prostaglandin have been reported in U.S. Pat. No. 3,826,823, which discloses dry preparation in which the prostaglandin is in a solid dispersion in PVP at solid state, and U.S. Pat. No. 3,954,787—Monkhouse, which discloses, lyophilized pharmaceutical composition containing prostaglandins, polyvinylpyrolidone PVP and succinic acid. Lypophilization provides also flexibility of dosage for the highly active prostaglandins.

U.S. Pat. No. 5,852,050—Okumura, et al. discloses a preparation, particularly a topical preparation, for the therapy of wounds or hemorrhoids, which contains, as an active ingredient, at least PGI2, PGE 1. Prostaglandin I2 and prostaglandin E1 are known to have broad range of pharmacological activities such as high inhibition activity of platelet aggregation and high stimulatory activity of vasodilating angiotelectasis and it has been expressed to apply these as drug against peripheral blood circulation impairments. Since, however PGI2 and PGE1 per see are chemically unstable, they are poor in retaining pharmacological effects and it is difficult to apply them to practical use.

U.S. Pat. No. 4,483,846—Koide discloses The present invention provides three-layered pharmaceutical film preparations that have a drug-storing middle layer composed of one or more (a) polyvinylpyrrolidone, (b) hydroxypropyl cellulose, (c) plasticizers and (d) organic acids, containing prostaglandin analogues, and two release-controlling layers on both sides of the said middle layer, composed of one or more (a) hydroxypropyl cellulose and (b) plasticizers, containing or not containing prostaglandin analogues, and which may release the drug at the desired concentration lastingly for an extended period of time, with great high biological availability, and can make this release “zero-order release”and further is said to improve stability. However, the form of the preparation is not retained at the administered site after administration.

The pharmacological properties of lyophilized prostaglandins, particularly PGE, are different from the base material and therefore the benefits typically associated with lyophilization are not available within this broad class of compounds. However, as disclosed in U.S. Pat. No. 3,826,823—O'Rourke, prostaglandins can be freeze-dried to give chemically and physically stable solid. Dry stabilized PVP-PGE preparation can be made by any suitable method, for example by mixing PGE solution in CO₂ and mixed with PVP powder. The ratio of prostaglandin to PVP can vary, depending upon the concentration of the prostaglandin desired in final in the final unit dose form, but the presently preferred range is one part of prostaglandin to about 10 to about 1000 parts PVP.

In general PVP provides a superior matrix for stabilizing the prostaglandin molecule. Reconstituted sample are tested for chemical stability. Regardless of the nature of the prostaglandin, its lyophilization and dispersion with the matrices of the selected excipients is stable and preferred form in which to store the prostaglandin prior to reconstitution and use by injection.

Lyophilization provides also flexibility of dosage for the highly active prostaglandins. U.S. Pat. No. 3,954,787. PG-E lyophilization on water-soluble polymer allows developing powder-containing alprostadil applied for wound healing. Lyophilized powder when contact with body liquid initially could release instantly some PGE, and forms instant gel containing PGE, which provides conditions for extended controlled release of alprostadil.

Thus, all currently developed products that contain PGE/alprostadil require refrigeration during storage because of poor stability of alprostadil at room temperature, a significant inconvenience particularly with relation to products directed to uses by consumers.

It is an object of the present invention to provide a preparation to provide topical preparations for the therapy, preferably wounds or hemorrhoids, or similar conditions that contain at least one of PGI2 ,PGE1 their derivatives and analogues thereof as an active ingredients, and a method for the therapy of wound or hemorrhoids or NTBD which comprises administrating the above active ingredient.

### SUMMARY OF THE INVENTION

The present invention relates to novel compositions of matter and methods for using same. More specifically the present invention is concerned novel compositions that include therapeutically effective amounts of prostaglandin, such as compounds of the PG-E type in anhydrous, water miscible, pharmacologically acceptable solvent systems. Most preferably, the active ingredient is provided in concentrations of at least 0.008 mg per milliliter (0.008 mg/ml). The present invention also relates to methods and apparatus for dispensing prostaglandin-E and similar compounds of the PG-E type. In preferred embodiments, these methods and apparatus comprise (1) dissolving the active ingredient, such as the prostaglandin compound, in an anhydrous, water-miscible pharmacologically acceptable solvent system; (2) packaging the resulting solution in unit dose containers, (3) diluting the contents of a container into a liquid or solid vehicle; and (4) administering the vehicle to administer a therapeutic dose.

The present invention relates to room temperature stable, non-aqueous Alprostadil (Prostaglandin E) compound dosage forms suitable for the treatment of sexual dysfunction. A stable solution of prostaglandin E—type compounds are obtained by dissolving the compounds in an anhydrous, water miscible, pharmacologically acceptable solvent system. It has been found that the stability of prostaglandins of the E group can be substantially enhanced without sacrificing bioavilability by the use of specific non-aqueous pharmacologically acceptable compositions. Preferably, the dose form is comprised of multiple components that are stored in separate compartments of a delivery package with a topical delivery vehicle and are combined prior to use.

The PGE-bearing formula can be prepared by first forming a solution of the desired PGE compound in a non-aqueous solvent such as C₂ to C₂₀ aliphatic alcohol e.g. methanol, ethanol, propanol, iso-propanol, n-butanol, t-butanol, pentanol and the like together with polymeric material, with or without a skin penetration enhancer. The resulting solution could be mixed with another substantially less volatile solvents such as PEG, Propylone glycols, hexylene glycols, or solutions of antimicrobial preservative , disinfectant agent like benzalkonium chloride, benzyl alcohol, phenylethyl alcohol, and bulking agents in a non-aqueous liquid solvent. Suitable bulking agent could be oil, white petrolatum, lecithin, lanolin, mineral oil polymeric material, silicones oils such as polydimethylsiloxane, e.g. cyclomethicone, dimethicone, silica, and the like; an optional alternative to the alkyl alcohol is alcohol containing aromatic ring like benzyl alcohol, phenethyl alcohol and the like together with permeation enhancers.

When mixture of liquids and solvents are applied with the preferred solvent content 0.5% to 75% by weight of the composition

Among the suitable stabilizer applied for the prostaglandin of the invention is an ascorbate. The preferred ascorbate for the invention is ascorbic acid or ascorbyl palmitate. The preferred concentration range for the stabilizer is from about 0.05% to 5% by weight of the mixture contained in the capsule comprising the invention, with the preferred being from 0.2 to 2%. Among the suitable penetration enhancer for use in dosage form could be cationic surfactant or mixture of cationic and anionic surfactant, organic acid and esters.

Other stabilizers may be incorporated along with the ascorbate in the mixture contained in the capsule. Among the other compounds that can act as these other stabilizer are included bisulfite, butylated hydroxyanizole (BHA), butylated hydroxy toluene (BHT), butylated hydroxyl quinine, thiodiproprionic acid, dilaurylthiodiproprionate, ethoxyquine, tocopherol, thiourea, thioglicerol, lecithin, propyl galate, nordihyroguairetic acid, 2 tert-butylhydroquinone, and hydroquinone, Triacetin, EDTA, edetate disodium, isopropyl myristate, vitamin E, and silica. The range of the other stabilizers can vary up to 5% by weight of the mixture contained in the capsule comprising the invention with the preferred being up to 0.2%.

The desired release rate, including controlled release or sustained release of the active compound can also be modulated by selection of the topical delivery vehicle, e.g., hydrophobic vehicle, white petrolatum, oil and the like. An optional alternative could be Carboxy-terminated gum or polyacrylic acid polymers, HPMC, PVP, cellulose derivatives (Carboxymethyl cellulose), gums, white petrolatum, mineral oil, oil, and silica suitable for use.

The ingredients listed above may be combined in any order or manner that produces a stable composition for ultimately receiving compound such as PGE₁, and the like, preferably substantially evenly dispersed throughout.

The present invention relates to devices for administering a pharmaceutical composition that have a first chamber containing a stable prostaglandin compound; and a second chamber containing a pharmaceutically compatible topical delivery vehicle, wherein upon administration, a pharmaceutical composition for topical application to a patient is delivered. The first chamber and second chamber can be separate portions of a single compartment or the first chamber and second chamber can each be a distinct compartment within a dual compartment structure.

The present invention also discloses a dosage form of a pharmaceutical composition comprising a sealed actives compartment containing a stable prostaglandin in a pharmaceutical composition for topical application to a patient. Preferably, the prostaglandin compound is sealed and substantially uniformly dispersed in delivery vehicle, and in certain embodiments the prostaglandin compound contains stabilizing agent comprising a moisture scavenger, an antioxidant and a chelating agent for metal ions. The stabilizing agent is preferably combined with prostaglandin compound. In certain preferred embodiments, the compartment contains at least one of the moisture scavenger, the antioxidant and the chelating agent for metal ions. The dosage can also include a material for enhancing the capacity of the prostaglandin to permeate skin.

The dosage form preferably also includes a delivery vehicle chosen from the group consisting of: gels, creams, liquid crystals, suspensions, and emulsions. It is preferred that the delivery vehicle has no substantial interaction with prostaglandin-E and any applied solvent, so that a monomeric form of prostaglandin-E is delivered to the patient.

In certain embodiments the delivery vehicle is preferably substantially anhydrous, free of metal ions and oxygen and water. It is also preferred that the composition can include a gelling agent dissolved in non-aqueous liquid, and additional constituents such as a surfactant and permeation enhancer. It is preferred that the delivery vehicle is water-soluble and provides release of drug upon contact with water, and that in certain embodiments other pharmacologically active substances are added that may have additive or synergistic activity including analgesic, anti-inflammatory, antimicrobial and antiviral agents. As explained above, in certain embodiments, the delivery material swells upon contact with water and thereby releases prostaglandin, and in certain embodiments the delivery vehicle forms a liquid crystal organized layer.

The improved compositions of the present invention are also useful for treating medical conditions by providing a packed mono or multi-component dosage form that comprises a sealed section containing an active pharmaceutical ingredient and a delivery vehicle pharmaceutically compatible for topical delivery. The medical condition can be, for example male or female sexual dysfunction broadly and male erectile dysfunction specifically. Alternately, the medical condition can be a wound or other conditions such as hemorrhoids. In preferred embodiments of the medical treatment methods the active pharmaceutical ingredient is prostaglandin, and most preferably, the prostaglandin is treated with a stabilizing agent comprising a moisture scavenger, an antioxidant and a chelating agent for metal ions. In certain embodiments, the prostaglandin dosage forms comprising: a volatile and non-volatile solvent, a non-volatile solvent and enhancer and an added enhancer.

In a specific application of the foregoing, a sock saturated with a gel forming material acting as lubricant is also disclosed which preferably has at least one wound healing composition to treat a foot skin condition, preferably using a wound healing composition such as prostaglandin.

### BRIEF DESCRIPTION OF THE DRAWINGS

FIG. 1 is a schematic representation of a two compartment device for delivering compositions made in accordance with the present invention; and

FIG. 2 is a schematic representation similar to FIG. 1 of a alternate embodiment of a two compartment device for delivering compositions made in accordance with the present invention.

### DETAILED DESCRIPTION OF THE PREFERRED EMBODIMENTS

The present invention relates generally to prostaglandin compositions. In particular, the present invention relates to storage stable, pharmaceutical compositions containing prostaglandins and enhancer. As used herein, the term “prostaglandin”or “PGE”refers to prostaglandins and derivatives and analogs thereof including pharmaceutically acceptable salts and esters, except as otherwise indicated by context.

Stability Enhancement

Co-pending provisional patent application Serial No. ______ , ______ filed on even date herewith entitled “Benzyl Alcohol Applications for Drug Delivery”and sharing the same inventive entity is incorporated herein by reference as if set froth in its entirety.

Prostaglandim and Surdactants

The present invention relates generally to prostaglandin compositions. In particular, the present invention relates to storage stable, pharmaceutical compositions containing prostaglandins and surfactants. As used herein, the term “prostaglandin”refers to prostaglandins and derivatives and analogs thereof including pharmaceutically acceptable salts and esters, except as otherwise indicated by context. Although certain embodiments are described with reference to PG-E, the invention is not limited to this particular form of prostaglandin.

Prostaglandin Composition

A number of prostaglandins that are useful as an active ingredient in accordance with the present invention are disclosed in U.S. Pat. No. 4,310,543—Gallo-Torres et al., which discloses soft gelatin capsules that contain a mixture comprising a solvent, either propylene glycol and polyoxyethylene sorbitan esters of fatty acids, a therapeutically effective amount of prostaglandin, and a chemical effective amount of ascorbic acid ascorbyl palmitate as a stabilizer. The prostaglandin composition disclosed is said to provide prostaglandin in bioefficient dosage form having improved stability and bioavilability and are said to be useful generally as anti-secretory agent.

As set forth in U.S. Pat. No. 4,014,989—Zaffaroni, solvents suitable for use with the prostaglandin compounds of the present invention include solvents that exhibit relatively higher degrees of polarity such as; tetrahydrofuran, chloroform, acetone, methylene chloride, ethylene chloride, dioxane, isobutyl ketone, methylisobutyl ketone, dimethyl ether, diethyl ether, alkanols, such as methanol, methyl butanol, n-amyl alcohol, 2-ethylhexyl alcohol, ethylene glycol, ethanol, isopropanol, hexanol, butanol, pentanol, and laser polar solvents such as benzene, carbon tetrachloride, cycloalkanes such as cyclopentane, 1,2dimethylcyclopentane, cyclooctane, isopropylcyclohexane, cyclohexane, and methylcyclohexane alkanes such as 3methylpentane, n-hexane, n-heptane and the like.

The mechanism of degradation of PGE is initiated by a variety of factors and can be prevented by applying appropriate steps to reduce or preclude degradation.

In accordance with the present invention, the degradation minimization technologies described herein can be applied to the formulation of the product containing PGE itself, or can be applied to solvent system and other components of the formula prior to formula preparation by providing PGE and a selected solvent, along with a gelling system. The resulting system demonstrates increased storage stability because PGE is inhibited from decomposition with water, oxygen and metal ions, as well as enzymes.

The selection of a solvent that is free of components that cause degradation, like moisture, oxygen or metal ions and enzymes assures maximum stability of PGE during storage in such a solvent system. The resultant solution exhibits an extended stability profile. Thus a stability kit could (1) be either incorporated directly into formula and assist the function of the product during storage as well application on tissue or (2) could be applied independently as a preventive pre-treatment of raw components for purpose removing degradation trigging elements from the raw materials. Solvent system pre-treatment and/or raw material pre-treatment is performed prior to the addition of the active compound, namely PGE. The benefits are that all these components can be eliminated from the formula itself and prevented from coming into contact with tissue. Excess applied solids for pre-treatment are easily separated from the liquid phase.

The present invention also provides new methods and apparatus for storing and administering therapeutically effective does of prostaglandin. In accordance with the formulations disclosed above. It is now possible to provide storage packaging for dry alprostadil that has either a single compartment in which a packed multi-component dosage form of sealed actives are mixed with delivery vehicle to create a pharmaceutically compatible for topical delivery. Alternatively, a dual compartment device can also be provided where one compartment contains solubilized drug or powder and a specially selected solvent that provides stable storage condition, while a second compartment contains a delivery vehicle so that when the components are mixed, a pharmaceutically compatible for topical delivery is created. The formulations can be dispensed, for example via pressurized containers using inert gas. Such containers might serve dual function: dispensing and also protecting from oxygen (increased stability) and can be constructed to protect the contents from exposure to light.

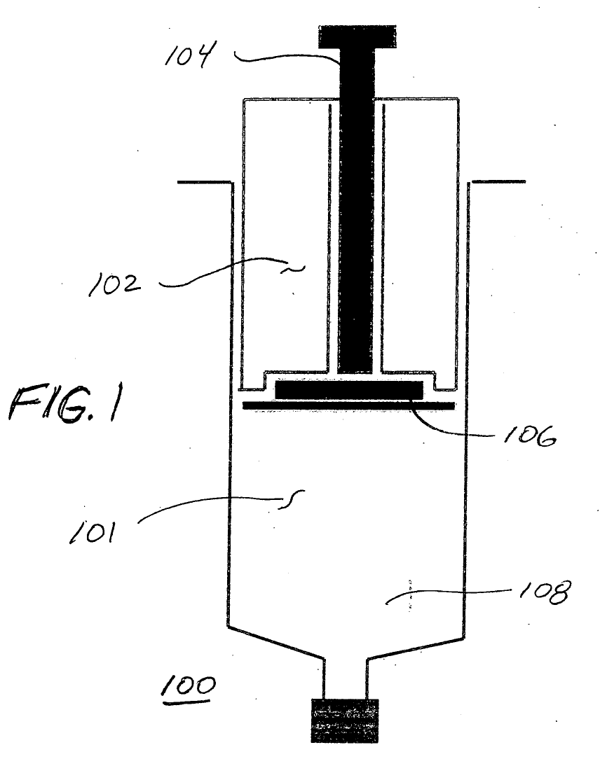

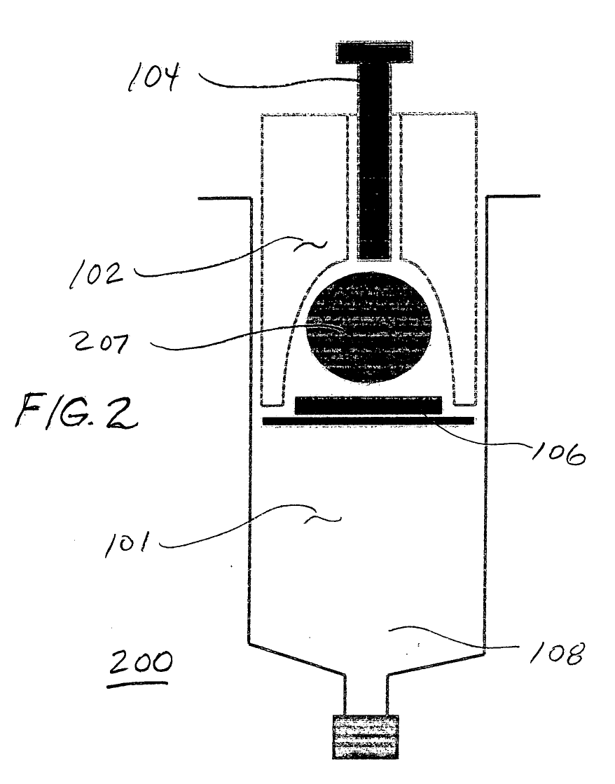

The present invention may be provided for use in two different types of devices or containers. In a first instance, all the ingredients can be incorporated into a dose unit for storage and treatment. In a second instance, raw material prepared with a triple components stability kit, discussed in further detail below is first prepared and then the drug, e.g., prostaglandin, is added in solution with a selected solvent. Referring now to FIG. 1, a first embodiment of a device useful with the present invention is shown. The device 100 is substantially in the form of a syringe and has two compartments, 101, 102 that are maintained separately. In use, the plunger 104 displaces the seal 106 so that the two materials in their respective chambers 101, 102 can be mixed. This step can be assisted by a mixing ball 108 if desired. FIG. 2 illustrates an alternate embodiment of a device 200 useful in conjunction with the present invention. In this illustration, like reference numerals refer to like features. Two chambers 101, 102 maintain the materials as separate, and are again mixed by displacing the plunger 104, however, in this embodiment the plunger acts upon a ball seal 207 that when displaced allows the materials to be mixed and further also displaces the seal 106, as described above. Those of skill in the art will realize that there are many ways to maintain two separate compartments in a device, and use the structure of that device to allow mixture of the materials when desired, for example, just prior to administration.

One preferred use for the present invention is a composition that may be used for topical applications in wound healing. Compositions and products made in accordance with the present invention will have unique characteristics, such as very low concentrations of PGE, in fact about 100 times lower, for this reason stability problem can also be addressed differently, for example by utilizing very low PGE solubility in delivery vehicle, or application of volatile solvent—no residual of organic solvent left in target formula In such embodiments, prostaglandins of different types, such as B, E, F, and I are administered.

In many embodiments of the present invention PGE is provided using lyophilization of neutral powder, however, in other embodiments, PGE is incorporated into an organized layer, and extended stability during storage results, as well as a controlled rate of release/solubility. Finally, interphase transfer is improved from the organized layer of the delivery vehicle into the organized layer of the lipid in the membrane, i.e., in the target tissue-intercellular material is made of organized lipids The stabilized prostaglandin compositions made in accordance with the present invention can be unit dose delivered from a storage container using a volatile solvent as a propellant, for example liquid CO2. The volatile nature of the propellant causes it to evaporate and thus not interfere with the pharmacological effects of the active ingredients delivered.

Thus, in several preferred embodiments, the present invention provides compositions for wound healing that are room temperature (RT) stable and body temperature stable so as to be useful for significantly longer than any compositions known in the prior art, i.e., for months or years. Preferably, the compositions are free of organic solvents and utilize a controlled release technology. Controlled release is obtained, for example, using a composition that forms organized layer in liquid crystal film with organized lamellar structure. This provides long lasting action and is advantageously combined with antimicrobial wound dressing. In certain embodiments, the compositions of the. present invention are delivered to the wound site without physical contact from an applicator, sponge, or the like, e.g., foam, emulsion, dispersion or spray deposits the composition on the wound site. Preferably, the delivery vehicle combined with the active ingredient is water-soluble and can include an ant-inflammatory and/or analgesic and/or antimicrobial/antiviral composition, known in the art, which creates temporary numbness at the wound site as a palliative measure to reduce discomfort and providing anti-inflammatory activity as well as anti-infective protection/treatment. Additionally, the wound healing composition could contain matrix components that contribute to the healing process by support collagen growth, such as vitamin E, lanolin, and the like.

As set forth above, certain preferred embodiments of the present invention use a composition that forms a liquid crystal film with organized lamellar structure to create a long lasting action. The benefits of an organized layer are that PGE part of liquid crystal network is prevented from degradation by keeping it isolated from other molecules. Another advantage is that the erosion of liquid crystal structure and liberation of PGE molecules achieve controlled release by gradual hydration. The liquid crystal also forms a protection membrane after exposure to water, but is washable from a wound or other application site prior to or following dressing. Finally, the liquid crystal layer trapping immobilized microorganisms. Liquid crystal layers useful with the present invention include techniques where the PGE is first solubilized and a liquid crystal formation agent with volatile solvent is added. Next a non-volatile solvent is added, such as benzyl alcohol or isopropyl myristate. The amount of volatile solvent is then reduced to the point that organized layer is formed and PGE become part of this structure. As will be understood by those of skill in the art, the performance of the resulting composition, e.g., the desired rate of release can be manipulated by adjusting the ratio of PGE and liquid crystal formation agent, e.g., a plasticizer. The resulting product formulation can then be blended into dose unit such as a film, dispersion, or cream. Preferably, liquid carbon dioxide is used as the volatile solvent under pressure and allows PGE to be incorporated into individual hydrophobic pockets of liquid crystal structure to create a new physical compound with new, and different properties of drug without such assistance (improved stability profile, rate of release). The water sensitivity of liquid crystal formation agents determine the rate of release of PGE and it is therefore an important consideration if there is wound washing prior to or following dressing.

In certain other embodiments of the present invention, mixing two components with diversified water affinity will be a valuable approach. Dispersion within a hydrophobic matrix phase is another approach.

In addition to wound healing, the present invention is useful for numerous topical applications such as vaginal delivery, hemorrhoid treatment, erectile dysfunction treatment, wound healing. The ability to provide long term room temperature storage at low concentrations permits a wide variety of prostaglandin compositions to be usefully delivered and to be commercially viable.

A preferred composition made in accordance with the present invention involves a dosage form of prostaglandin that has a stabilizing kit/agent made from a moisture scavenger, an antioxidant and a chelating agent for metal ions. Such a composition has better stability profile than the compositions known in the prior art will be room temperature stable for 1-2 years or more. Other elements may be included in the stability kit, such as silica, for water removal

In a preferred embodiment, the stable compositions of the present invention are combined with a carrier that allows either a single coating of an active ingredient to be applied to the wound site, or more preferably, a two coat system wherein the first coat is covered with a protective coating to enhance the wound healing process.

Preferred embodiments of the wound healing application of the present invention have PGE concentrations of about 0.001-0.01% w/w and most preferably about 0.003% w/w. Since the low solubility of the active ingredients in water is sufficient to provide wound treatment. For example, PGE has solubility 0.008% in water and a typical concentration in wound healing cream of about 0.003%.

In one preferred formulation, lyophilized PGE powder is delivered on an insoluble solid by coating the solid with PGE and the body fluid provides water to solubilize the PGE sufficient to treat the wound.

As set forth above, one preferred method of application involves spraying or other techniques that do not require contact with the wound surface itself and does not disrupt the natural healing process nor irritate the nerve endings. Upon initial application, as described above, the aqueous nature of tissue fluid will release PGE or other active ingredients from applied formula and begin drug interaction with the wound tissue. In certain embodiments, there will also be delayed delivery of the active compound under a rate-controlled mode.

The composition of the product can provide several beneficial interactions, including isolation against microbial and antimicrobial protection. As will be understood by those of skill in the art, longer-term wound care requires debridement, and other concerns regarding wound maintenance need to be addressed. Therefore, the compositions of the present invention are either re-applied or must otherwise account for other necessary wound care procedures. For example, a secondary protection layer applied after certain time by separate spray will prevent the active ingredient from being debrided, or drug partitioning between wound tissue and delivery vehicle needs to be design such way, that continue delivery of drug into wound tissue will take place with time after treatment application.

One know drug delivery excipient is chitosan, which forms a gel at pH values below 5.5 and is useful for delivering PGE in accordance with he present invention.

Long lasting skin exposure to friction and abrasion is inevitable during extensive daily activity. However under extreme conditions, this could be a problem, particularly in long lasting exposure to moisture, soil dust or mud abrasion process is highly accelerated and leading not only to the skin damage by friction, but also to performance limitation or complete elimination. People exposed to intensive walking are experience of foot skin damage by abrasion in specific area like heel, or knuckle area. Frequent occurrence of skin damage could be performance-limiting factor. The most sever outbreak are generally associated with tropical climates and exposure to new strain of fungi. The medical report on skin disease in British Army shows that 34% of European soldiers in Mediterranean theater were reported. Vietnam data from tropical climate shows significant dermatophytosis on incidence of 65%.

Thus, it is known that extended exposure to moist environment stimulates fungal invasion in hiking shoes, laborers/workers shoes, military and law enforcement personnel, athletes and many other situations. Reducing these conditions will provide prevention and safety irnprovements. The treatment option for the management of cutaneous fungal infections is becoming particularly important to maintain skin in good functional conditions.

It would therefore be desirable to provide foot skin protection against such damage, as well as provide treatment in early and advanced stage of damage. Thus, in accordance with this aspect of the present invention socks are created from a material saturated with a composition that, under typically encountered environmental conditions start to form a gel with the capacity to release active ingredients. In typical environmental conditions (high heat, high humidity) this gel preferably provides several functions, namely:
Wear protection by reducing friction, i.e., a lubricity effect
Reduced microbial activity by immobilization and killing of the fungi and fungi causal properties
Release of a wound healing drug from gel layer
Lack skin porous structure by lipids to make more durable, Lipids barrier reduced water permeation by lipid deposit+lubricity effect.

In certain embodiments not all of the foregoing characteristics will be present, while in other embodiments, and in accordance with the other aspects of the present invention set forth above, formulation such as the prostaglandin formulation disclosed herein can be advantageously incorporated into the sock and/or into the gel.

In preferred embodiments, the socks are elastic woven socks that have the capacity to adjust to various foot shapes and thereby provide a protection layer that additionally adheres to the skin in selected areas by applying heat sensitive or moisture sensitive adhesives in order to prevent movement against skin when contact with footwear. Therefore, displacement and abrasion action will take on the interface between the protective layer and footwear, not between the wearer's skin and the socks. Transferring the rubbing action to the external interface of the socks significantly reduces skin damage. Applying extra treatment with a lubrication agent to skin and/or to footwear will significantly reduce wearing process for skin. Frictional displacement at the interface is major factor causing intensive damage for skin. Therefore, in accordance with the invention, a gel-forming material is incorporated inside a woven, porous capillary structure. The sock coating with gel forming material is preferably either applied to the entire sock or only to selected zones and depends on desired effect. Gel forming material also could be applied in specific pattern to maintain membrane functions; breathable for air oxygen and moisture.

After early detection of skin damage and instant application of protection layer would assure/improve healing process, as described above with relation to other aspects of the present invention. In particular, reduced skin barrier function by abrasion increases the probability of microbial invasion of the skin. Applying protection layer in this area is highly desired to recover/ improve barrier function as well as to provide assistance in healing process.

In accordance with the present invention, a combination of antifungal and wound healing agents will serve these special needs. Controlled release of both actives from a gel forming layer assures the desired antifungal protection and provides support for wound healing. Presence of a gel layer in such areas will provide a barrier function as well and further provides protection against skin abrasion.

In certain regards, the socks described herein and the gel forming layer created by their use could be considered as a second skin with desired protection functions against friction. The protection layer may contain medical therapeutic agent for wound healing and antifungal agent as well as non-medical ingredient well known with wound healing capacity.

A variety of materials are useful in these embodiments. For example, HPMC is well known for control release capacity, and chitosan and cationic materials with antimicrobial capacity are all useful for the protection layer. Cationic materials are well known for skin adhesion/affinity, many of them provide antimicrobial capacity. Silicon based compounds are useful as wear protection as well as a controlled release vehicle.

Lanolin is well known for heavy odor, recently upgraded product to medical applications is also known as wound healing agent. The hydrophobic nature of this material makes it a good provider for lubricity, and similarly white petrolatum increases the lipids barrier. Another class of gel forming material is silicon dioxide. Finally, other materials that either enhance the mechanical frictional properties or, like vitamin E that provide medical benefits such as supporting wound healing growth factors can also be included in the composition.

Although the present invention has been described in accordance with the embodiments shown these descriptions are provided in order to fully describe the present invention and are not limiting. One of ordinary skill in the art will readily recognize that there are numerous variations to the embodiments and those variations would be within the spirit and scope of the present invention. Accordingly, many modifications may be made by one of ordinary skill in the art without departing from the spirit and scope of the appended claims. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable arts. The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Accordingly, reference should be made to the appended claims in order to ascertain the true scope of the present invention.

What is claimed is:

1. A device for administering a pharmaceutical composition comprising a first chamber containing a stable prostaglandin compound; and a second chamber containing a pharmaceutically compatible topical delivery vehicle, wherein upon administration, a pharmaceutical composition for topical application to a patient is delivered.

2. The device of claim 1, wherein the first chamber and second chamber are separate portions of a single compartment

3. The device of claim 1, wherein the first chamber and second chamber are each a distinct compartment within a dual compartment structure.

4. A dosage form of a pharmaceutical composition comprising a sealed actives compartment containing a stable prostaglandin in a pharmaceutical composition for topical application to a patient.

5. The dosage form of claim 4, wherein the prostaglandin compound contains stabilizing agent comprising a moisture scavenger, an antioxidant and a chelating agent for metal ions.

6. The dosage form of claim 4, further comprising a material for enhancing the capacity of the prostaglandin to permeate skin.

7. The dosage form of claim 4, wherein delivery vehicle is chosen from the group consisting of: gels, creams, liquid crystals, suspensions, and emulsions.

8. The dosage form of claim 4, wherein the delivery vehicle has no substantial interaction with prostaglandin-E and any applied solvent, whereby a monomeric form of prostaglandin-E is delivered to the patient.

9. The dosage form of claim 4, further comprising a gelling agent dissolved in non-aqueous liquid.

10. The dosage form of claim 4, further comprising one or more additional pharmacologically active substances exhibiting one or more of analgesic, anti-inflammatory, antimicrobial and antiviral activity.

11. The dosage form of claim 4, wherein the delivery material swells upon contact with water and thereby releases prostaglandin.

12. The dosage form of claim 4, wherein the delivery vehicle forms an organized layer substantially in the form of a liquid crystal, whereby the resulting composition has controlled release capacity.

13. A method treating a medical condition comprising providing a packed mono or multi-component dosage form that comprises a sealed section containing an active pharmaceutical ingredient and a delivery vehicle pharmaceutically compatible for topical delivery.

14. The method of claim 13, wherein the medical condition is sexual dysfunction.

15. The method of claim 14, wherein the sexual dysfunction is male erectile dysfunction.

16. The method of claim 13, wherein the medical condition is a wound.

17. The method of claim 13, wherein the active pharmaceutical ingredient is prostaglandin.

18. The method of claim 17, wherein the prostaglandin is treated with a stabilizing agent comprising a moisture scavenger, an antioxidant and a chelating agent for metal ions.

19. A sock saturated with a gel forming material acting as lubricant and controlled release.

20. The sock of claim 21, further comprising at least one wound healing composition to treat a foot skin condition.
